(19) United States
(12) Patent Application Publication
(10) Pub. No.: US 2015/0238956 A1
(43) Pub. Date: Aug. 27, 2015

(54) CONICAL MULTI-WELL FILTER PLATE

(71) Applicant: Centre Hospitalier Universitaire Vaudois, Lausanne (CH)

(72) Inventors: Eric Grouzmann, La Conversion (CH); Marielle Dunand, Prevessin-Moens (FR); Danilo Gubian, Chardonne (CH)

(73) Assignee: CENTRE HOSPITALIER UNIVERSITAIRE VAUDOIS, LAUSANNE (CH)

(21) Appl. No.: 14/427,130

(22) PCT Filed: Sep. 11, 2013
(86) PCT No.: PCT/IB2013/058457

§ 371 Date: Mar. 10, 2015

Related U.S. Application Data
(60) Provisional application No. 61/699,522, filed on Sep. 11, 2012.

(51) Int. Cl. B01L 3/00; G01N 30/02; B01D 15/10

(52) U.S. Cl. CPC B01L 3/50255; B01D 15/10; G01N 30/02; G01N 2030/027

(57) ABSTRACT

The present invention relates to a multi-well filter plate able to facilitate, simplify and accelerate preparation and treatment of samples to be analyzed by for example liquid chromatography tandem mass spectrometry (UPLC—MS/MS method for simultaneous quantification of analytes).

### FIELD OF THE INVENTION

The present invention relates to a multi-well filter plate able to facilitate, simplify and accelerate preparation and treatment of samples to be analyzed by for example liquid chromatography tandem mass spectrometry (UPLC—MS/MS method for simultaneous quantification of analytes).

### BACKGROUND OF THE INVENTION

Plasma catecholamines including epinephrine (E), norepinephrine (NE) and dopamine (DA) are helpful markers to evaluate adrenosympathetic function in patients and animal models. In addition, quantification of catecholamine concentrations in plasma is considered clinically important for the diagnosis of pheochromocytoma and paraganglioma during dynamic clonidin tests. Because of their low concentrations in plasma, the instability of the catechol group and the small sample volumes collected from preclinical models such as mice and potential chromatographic interferences from compounds that co-elute with catecholamines and are oxidized make accurate and fast measurement of plasma E and NE still a challenge.

Several analytical methodologies have been reported associating fluorimetric, spectrophotometric, radioenzymatic, gaz chromatography and high performance liquid chromatography (HPLC) techniques for quantitative analysis of plasma catecholamines. HPLC with electrochemical detection (HPLC-EC) is now recognized as the gold standard method because of its high sensitivity (limit of detection less than 0.2 nmol/1 in 25 microliters of sample and selectivity for the analysis of catecholamines but it requires time-consuming and complex sample preparation and long chromatographic runs to reduce analytical interferences with co-eluting analytes that exhibit redox behavior similar to catecholamines.

The most popular sample preparation method relies on the properties of activated aluminum oxide to retain catecholamines at a basic pH of 8.5 and elution at acid pH with a yield recovery of 60-80%.

Tandem mass spectrometry methods (MS/MS) interfaced with UPLC have been reported with the advantage to offer higher analytical specificity because detection is based on the retention time, the molecular mass and chemical structures, properties unique to each molecule. However, these methods suffer from several limitations to reach the desired sensitivity in small sample volumes obtained in mice and because catecholamines are very polar and not easely ionizable. Catecholamine in spiked plasma samples have been separated and detected by applying LC/MS with the high limit of detection around 5000 nmol/1 for NE, E and DA. To circumvent this problem, a method associating the combination of UPLC—MS/MS with a reductive ethylation technique has been proposed in order to reach lower limit of quantification of systemic concentrations of E and NE at 0.27 and 0.30 nmol/1 in 25 μl of plasma. However, the reductive ethylation labeling of catecholamines must be carried out in a fume hood since it uses sodium cyanoborohydride, a highly toxic chemical that will produce hydrogen cyanide gas when exposed to acid.

Thus there is still a need to find a simple, safe and rapid analytical system to determine analytes in liquid samples, such as blood plasma samples.

### SUMMARY OF THE INVENTION

Applicants have designed a specific multi-well filter plate able to facilitate, simplify and accelerate preparation and treatment of samples to be analyzed by for example liquid chromatography tandem mass spectrometry (UPLC—MS/MS method for simultaneous quantification of analytes).

Thus the present invention provides a multi-well filter plate comprising a base plate having a plurality of wells therein, whereas each well of said plurality of wells is conical, with its wide end forming an inlet to said well on an upper side of said base plate and its narrow end being oriented towards a bottom side of said base plate, said well further comprising a filtered outlet for filtering and draining content out of said well.

The present invention further provides a multi-well test apparatus comprising the multi-well filter plate of the present invention and a feeding tray supporting said filter plate, said feeding tray having an inclined support surface comprising:
a drainage area from which liquid can be removed and an introduction area into which liquid can be supplied, said inclined support surface being inclined in a configuration to effect drainage of liquid from said introduction area to said drainage area, and
walls surrounding said inclined surface to enclose said inclined surface.

The present invention also provides a kit comprising the multi-well filter plate according to the invention and chemical reagents.

### BRIEF DESCRIPTION OF THE FIGURES

FIG. 1 shows a cut of a micro-well of a multi-well plate according to an embodiment of the invention;

FIG. 2 shows a partial cut of the base plate of the multi-well plate of FIG. 1;

FIG. 3 is a detailed cut view of the cap closing the outlet of the micro-well of FIG. 1;

FIG. 4 is a cut view of the filter support of the micro-well of FIG. 1;

FIG. 5 is a cut view of the outlet of the micro-well of FIG. 1;

FIG. 6a shows chromatogram from a blank;

FIG. 6b shows chromatogram from a healthy subject;

FIG. 6c shows chromatogram from an example of a patient with histologically confirmed pheochromocytoma;

FIG. 7 shows no discernable ion suppression at the retention times for E, NE and DA;

FIG. 8a represents Deming regression curves of plasma E concentrations measured by HPLC-ECD (gold standard) and by UPLC-MS/MS methods;

FIG. 8b represents Deming regression curves of plasma NE concentrations measured by HPLC-ECD (gold standard) and by UPLC-MS/MS methods;

FIG. 8c represents Deming regression curves of plasma DA concentrations measured by HPLC-ECD (gold standard) and by UPLC-MS/MS methods;

FIG. 9a shows Altman Bland plots representations of mean difference between HPLC-ECD and UPLC-MS/MS for E;

FIG. 9b shows Altman Bland plots representations of mean difference between HPLC-ECD and UPLC-MS/MS for NE;

FIG. 9c shows Altman Bland plots representations of mean difference between HPLC-ECD and UPLC-MS/MS for DA;

FIG. 10 shows calibration curve obtained with immunoextraction of parathormone 1-34 (PTH1-34) from a plasma matrix

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

As used herein, the term “comprise” is generally used in the sense of include, that is to say permitting the presence of one or more features or components.

As used in the specification and claims, the singular forms <<a>>, <<an>> and <<the>> include plural references unless the context clearly dictates otherwise.

The present invention provides a multi-well filter plate comprising a base plate having a plurality of wells therein, whereas each well of said plurality of wells is conical, with its wide end forming an inlet to said well on an upper side of said base plate and its narrow end being oriented towards a bottom side of said base plate, said well further comprising a filtered outlet for filtering and draining content out of said well. Preferably said filtered outlet comprises a filter. Also preferably said filtered outlet comprises a membrane filter and a filter holder for holding said membrane filter at the narrow end of said well.

The filtered outlet can further comprise an outlet tube for forming droplets of a liquid flowing out of the well through the filtered outlet. The filtered outlet can further comprise a cap for preventing liquid from flowing out of the well through said filtered outlet.

The multi-well filter plate according to the present invention can further comprise a cover for covering the inlets of said plurality of wells for preventing analytes from flowing out of said wells when said well is moved for mixing said analytes inside said plurality of wells.

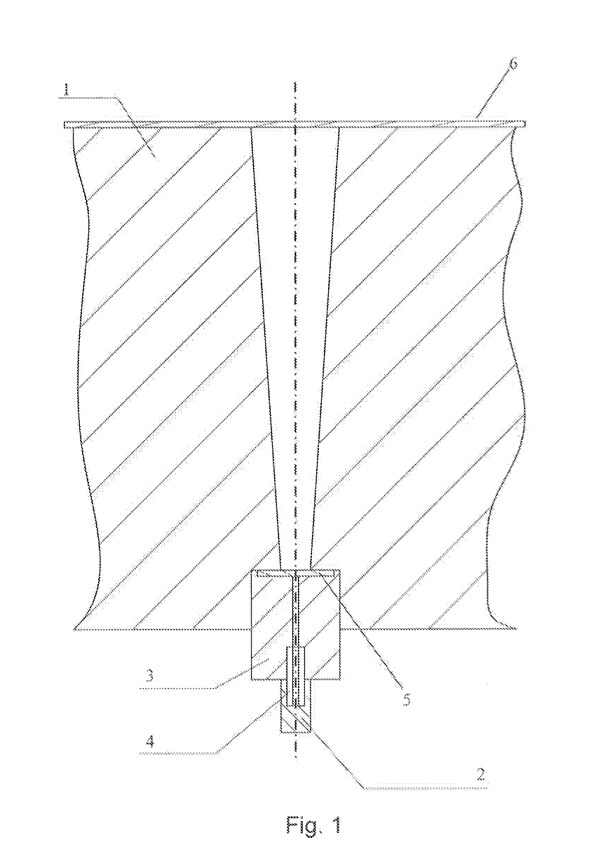

With reference to FIG. 1 showing a partial cut of a multi-well plate according to an embodiment of the invention, the multi-well plate of the invention comprises a base plate 1, for example a plate of plexiglass or any other appropriate material, and a plurality of conical micro-wells formed in the base plate 1. For the sake of readability, only one micro-well is visible in the figures. The micro-wells are preferably oriented with their wide end towards the top, or upper surface of the base plate 1 and their narrow end towards the bottom of the base plate 1.

A plurality of inlets, each to one of said plurality of micro-wells, is thus formed on the upper side of the multi-well plate, thereby allowing for example the insertion of samples, for example liquid biologic samples, into the micro-wells.

In embodiments, each conical micro-well further comprises an outlet on their narrow, lower end, exiting on the bottom, or lower side of the base plate 1. The outlet is for example a filtered outlet comprising a filter 5, for example a membrane filter, and a filter support 3 for holding said filter 5 against the lower end of the conical micro-well. The filter support 3 is for example made of Polymethacrylate methyl (PMMA) or any other adapted material, and at least partly inserted and frictionally held into an adapted recess in the lower side of the base plate 1. The filter support 3 is for example cylindrical and at least partly inserted in a cylindrical bore formed on the bottom side of the base pate 1 and coaxially aligned with the corresponding conical micro-well.

Preferably the microporous membrane filter is selected from the group comprising nitrocellulose membrane, cellulose membrane, cellulose acetate membrane, polycarbonate membrane, polyvinylidene fluoride membrane and polysulfone membrane.

Other embodiments are however possible within the frame of the invention for holding the filter within the bottom part of the conical micro-well. For example, a single filter support can be configured for supporting the filter within two or more micro-well simultaneously, possibly within all micro-wells of the multi-well plate. Accordingly, the filter support comprises two or more protruding parts that are inserted into corresponding recesses in the bottom side of the base plate 1.

According to embodiments of the invention, the filter 5 is firmly held between the filter support 3 and a shoulder formed at the junction between the conical micro-well and the recess in which the filter support 3 is inserted. Preferably, the filter support 3 comprises an opening for allowing the filtrated elements to exit the micro-well. The opening is for example coaxially aligned with the micro-well when the filter support is inserted in the recess. In the illustrated example, the filter support is a cylindrical element and the opening is located in the centre of the filter support, along its longitudinal axis

In the illustrated exemplary embodiment, the filtered outlet further comprises an outlet tube 4 that is inserted in the filter support 3 for forming droplets of a liquid exiting the filtered outlet, and a removable cap 2 for closing or opening the filtered outlet. The cap 2 is made for example of silicone or any other adapted material. The tube 4 is made for example of polyetherether-ketone (PEEK) or any other adapted material. In the illustrated exemplary embodiment, the outlet tube 4 protrudes from the filter support 3 and the cap 2 is frictionally held on the protruding end of the outlet tube 4. Other embodiments are however possible within the frame of the invention for controlling the outflow from the micro-well and/or the closing and opening of the filtered outlet.

The filter 5, for example a membrane filter, is for example made in porous PTFE (e.g. Macherey-Nagel, Porafil membranes, pore size 3 μm, diameter 6 mm, ref 670300013) or PE (e.g. Macherey-Nagel, Porafil membranes, pore size 5μm, ref 671500013), for retaining for example activated aluminum oxide resulting from the solid phase extraction (SPE) of plasma catecholamines Other filter material, for example adapted to other application, can however be used within the frame of the invention.

In embodiments, the top of the well can be closed during mixing, for example by a plate sealer 6 (e.g. Promega AG, ref 5701) or by a cap foil of serigraphic quality (e.g. ref. Spondex WKU310), or by any other suitable element.

The multi-well plate for example comprises 96, 192, 384 or 768 micro-wells.

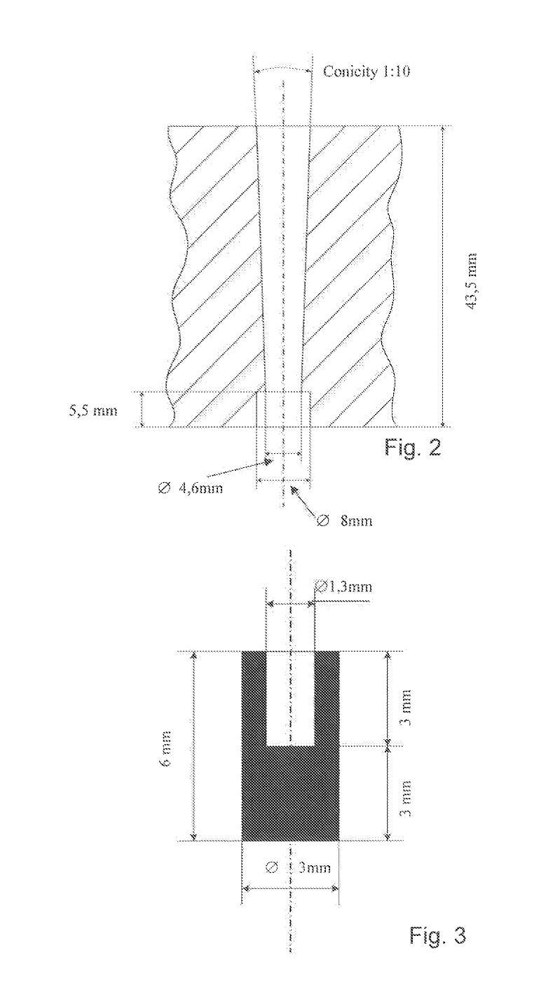

FIGS. 2 to 5 show detailed cut-views of single elements of the micro-well plate with quotes. The illustrated embodiments, and in particular their indicated dimensions, are purely illustrative and in no way limiting. In particular, the dimensions can be varied, for example to adapt the micro-well plate of the invention to various applications and/or to manufacturing constraints, material limitations, etc.

FIG. 2 is a partial cut view of a base plate 1 according to an embodiment of the invention, showing one conical micro-well formed therein, with its widest opening on the upper side of the base plate 1, and a recess in the lower side of the base plate 1, for inserting a filter and a filter support for forming a filtered outlet to the micro-well. In the illustrated exemplary embodiment, the recess is a cylindrical bore that is coaxially aligned with the conical micro-well. Other configurations of the recess are however possible within the frame of the invention.

FIG. 3 is a cut view of an embodiment of a cap 2 that can be removeably placed at the outlet of the micro-well for controlling the outflow of filtered fluid from the micro-well. In the illustrated exemplary embodiment, the cap 1 is a cylindrical element with a central blind hole. The cap can for example be placed over the tubing protruding from the filtered outlet of the micro-well. Other configurations of the cap are however possible within the frame of the invention.

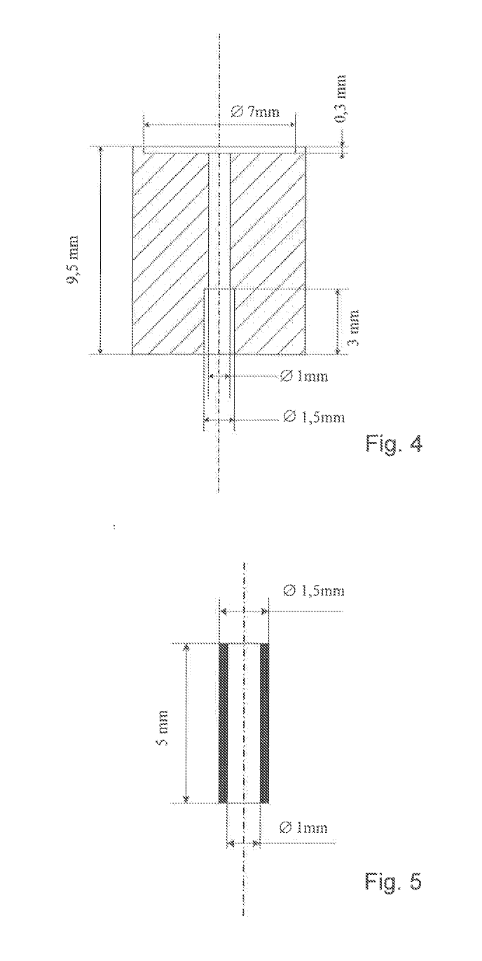

FIG. 4 is a cut view of an exemplary embodiment of a filter support 3. The filter support 3 is for example a cylindrical element with a centre opening along its longitudinal axis for allowing fluid to flow therein, for example filtered fluid flowing out of the corresponding micro-well. The filter support 3 further comprises a recess on a face around an extremity of the centre opening for lodging a filter, for example a membrane filter. In the illustrated example, the centre opening is made slightly wider on a determined length along its end opposite the recess for lodging the filter, for receiving an outlet tube for forming droplets of the liquid flowing through and exiting the centre opening through the outlet tube. Other configurations of the filter support are however possible within the frame of the invention.

FIG. 5 is a cut view of an exemplary embodiment of an outlet tube 4 for use in a filtered outlet according to embodiments of the invention. The outlet tube is a piece of tube with dimensions adapted for its at least partial insertion within an opening of a filter support of the filtered outlet, and for forming droplets of a liquid flowing through it, for example under the effect of the gravity force.

According to the invention, the conical shape of the micro-wells allows the creation of a vortex by agitation of the multi-well plate similar to what can be obtained by agitation of a microcentrifuge tube in a vortex.

Applicants designed a multi-well plate for solid phase extraction (SPE) to isolate analytes of interest from a wide variety of matrices, such as urine, blood, water, beverages, organic solution, soil and tissues (such as human, animal or plant tissues).

In the context of the present invention, SPE uses the affinity of analytes dissolved or suspended in a liquid sample (mobile phase) for a solid (stationary phase) when analytes are contacted with a solid, to isolate desired analytes and separate them from other components present in a liquid sample. Said solid (stationary phase) can be for example activated aluminum oxide, immunoconjugates, chelates, immunoaffinity beads, etc. The desired analytes of interest in the sample are retained on the stationary phase, which is retained in wells of the multi-well filter plate of the invention by filtered outlet. The portion of the liquid sample, which does not contain the analyte of interest, passes through the filtered outlet and can be discarded. The analyte of interest retained on the stationary phase can then be recovered from the stationary phase as eluate. The eluate resulting from the desorption (recovery) of the analyte from the stationary phase passes through the filtered outlet for collection in a microplate for further analysis, whereas the stationary phase is retained in the wells.

One advantage of the multi-well plate of the present invention is that the stationary phase is not coated (fixed) on the walls of the wells, but is free in the solution, which allows better capture of analytes by agitation (vortexing). Another advantage, is that there is no need to transfer the eluate resulting from the desorption (recovery) of the analyte from the stationary phase into another vial since this procedure operates directly within the wells, minimizing losses linked to pipeting of microliters.

The multi-well plate according to the invention can be used for solid phase extraction (SPE) of catecholamines. Thanks to the filtered outlet the activated aluminum oxide is retained in the bottom of the micro-well and liquids remain above the filter as long as the cap is inserted on the outlet tube 4. Once the cap 4 is removed, the liquids drain out of the micro-well through the filter after vacuum is applied and the outlet tube 4, thereby forming small droplets that will fall down, for example in a collection plate, after elution of the catecholamines.

Quantification of catecholamine in plasma provides a reliable biomarker of sympathetic activity and is useful for the diagnosis of pheochromocytoma. The low circulating concentrations of norepinephrine (NE) and especially epinephrine (E) and dopamine (DA) and analytical interferences require tedious sample preparation and long chromatographic runs to ensure their accurate quantification commonly by HPLC with electrochemical detection.

A 96-well filter plate containing activated aluminum oxide is used to remove interfering substances and extract catecholamines prior to UPLC MS/MS analysis. The multi-well plate is able to simultaneously quantify catecholamines in 50 to 250 μl of plasma. According to this embodiment, plasma samples are introduced in the conical wells of the multi-well plate comprising 96 microwells specially designed to allow efficient mixing of the solution with activated aluminum oxide. The liquid remains in contact with the alumina by thorough mixing for 15 minutes and the plate is disposed on a vacuum pumping system to drain the liquid out of the micro-wells and through their filtered outlets while the activated aluminum oxide is retained onto the filter, for example a 3 μm porous membrane placed at the bottom of the conical well. After 3 washes with water the catecholamines are eluated with water containing formic acid on a collecting plate and directly injected into the UPLC tandem MS TQD. Each analyte is quantified based on the MRM signature of daughter ions and recovery is ensure using commercially available isotopic standards.

This method presents several advantages compared to other LC-MS/MS methods since it does not require derivatisation of catecholamines prior analysis, it offers high throughput with the multi-well microplate designed to capture catecholamines directly on activated aluminum oxide and eluate them on collecting microplates to reach high sensitivity, plasma catecholamine concentrations in patients and healthy volunteers were similar to values obtained with HPLC methods, which used electrochemical detection. This method is reliable, reduces turnaround time for routine application and adequately sensitive to obtain measurements of plasma catecholamines in small sample volumes from mice and children. Indeed processing time, which included sample purification on activated aluminum oxide and elution is less than 1 h per 96-well plate. UPLC—MS/MS analysis run time is 2.0 min per sample. The lower limits of quantification were 0.05 nmol/L for E, 0.25 nmol/L for NE, and 0.15 nmol/L for DA. The linearity of this method was excellent within the whole calibration range from 0.02 to 6.2 nmol/L (r²>0.98). We found a positive matrix effect for each analyte with a process efficiency ranging from 84 to 99%. The intra-run and inter-run assay coefficient of variations ranged from 3 to 23.9% and 0.9 to 13.8%, respectively. Deming regression of HPLC-EC and UPLC-MS/MS results yielded slopes of 1.02 for E, 0.92 for NE and 0.77 for DA and y-intercepts of −0.05 for E, 0.22 for NE and 0.15 for DA. Reference intervals for 120 healthy subjects were 0.03-0.98 nmol/L (E), 0.63-4.51 nmol/L (NE) and <0.33 nmol/L for DA.

The use of the multi-well filter plate as per the present invention allows a correct mix of the activated aluminum oxide with plasma on the top of a polyethylene filter prior applying vacuum to discard flow-through and eluate the catecholamines after two wash step into a collecting multi-well plate suitable for direct injection into the UPLC-MSMS system without the need for solvent evaporation. This result is increase in throughput for extraction and turn around time for analysis. The conventional method by HPLC-ECD for extraction of plasma catecholamines requires for 24 extractions 3 hours of sample handling and the HPLC analysis 30 min per sample representing 3 hours+12 hours=15 hours=900 min/24 samples=37.5 min/sample. In contrast, UPLC tandem MS method and the multi-well filter plate as per the present invention requires 50-250 microliters of plasma and several extractions, for example 96) may be done in 2 hours, UPLC tandem MS analysis requires 2 min per sample (192 min=2 h30 min) making the whole process duration at 2:30 hours +2 hours=270 min/96 samples=3 min/result. Decrease of volume of plasma necessary for the analysis is also an advantage for microsamples in pediatric patients and preclinical research in small animals such as rodents.

Another application of the multi-well plate according to the present invention is for immunoextraction of bioanalytes. Biological samples containing the analyte and an immunoconjugate are introduced in the wells of the multi-well filter plate and a vortex is generated by agitation of the multi-well filter plate. Once the immunocapture of the analyte has been performed, the liquid is removed from the wells by vacuum, whereas the complex immunoconjugate-analyte remains in the wells. After the wash step, the analyte is removed from the immunoconjugate and directly microeluated in a collecting microplate for further analysis.

The advantage of the multi-well filter plate of the present invention is that a large number of samples may be processed simultaneously and recovered in a small volume for further processing.

The multi-well filter plate of the present invention may be used with various analytes and solid phase extraction procedures, such as immunoaffinity beads and chelators.

In a further embodiment, the present invention provides a multi-well test apparatus comprising the multi-well filter plate of the present invention and a feeding tray supporting said filter plate, said feeding tray having an inclined support surface comprising:
a drainage area from which liquid can be removed and an introduction area into which liquid can be supplied, said inclined support surface being inclined in a configuration to effect drainage of liquid from said introduction area to said drainage area, and
walls surrounding said inclined surface to enclose said inclined surface.

In another embodiment, the present invention provides a kit comprising the multi-well filter plate according to the present invention and chemical reagents. The kit can further comprise a cover. The kit can also further comprise a collecting microplate that is located along the bottom of the multi-well filter plate, said waste tray element being adapted to receive filtrate from the multi-well filter plate.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Example 1

Patient Samples

Reference Values in a Normal Population

120 healthy normotensive volunteers (aged 22 to 56 yr) of both sex were instructed to fast overnight and to abstain from caffeinated beverages. Subjects that consume antidepressant were excluded from the study. Five ml blood sample (containing Li heparinate) was collected after 20 min of supine rest, immediately centrifuged at 4° C. and the plasma stored at −80° C. until analysis. This study has been approved by the local ethics committee.

### Chemical Reagents

Sodium metabisulfite (Na₂S₂O₅), Trizma® base (Tris (hydroxymethyl)aminomethan), (±)-Epinephrine hydrochloride, (±)-Norepinephrine (+)-bitartrate salt and Dopamine hydrochloride were purchased from Sigma-Aldrich, Buchs, CH. Titriplex® III (ethylenedinitrilotetraacetic acid disodium salt dehydrate), Hydrochloric acid 37% (HCl) were obtained from Merck, Dietikon, CH. Water (ULC/MS); Metanol (ULC/MS) and Formic acid 99% (ULC/MS) were supplied from Biosolve, Basel, CH. aluminum oxide was obtained from

Macherey-Nagel, Oensingen, CH. (±)-Epinephrine-2,5,6,α,β, β-d6 (d6-E) (99.1 at ²H), (±)-Norepinephrine-2,5,6, α,β,β-d6 (d6-NE) HCl (98.7 at ²H) and Dopamine-α,α,β,β-d4 (d4-DA) HCl (99.6 at ²H) were ordered From Alsachim (Illkirch Graffenstaden, France).

Preparation of Calibration Curves and Quality Controls

Calibration curves were prepared by serial dilutions 1:2 (v/v) with blank plasma of a certified plasma calibrator for plasma catecholamines from Chromsystems, München, Germany (ref. 5009). Blank plasma was obtained from a pool of heparin-lithium plasma depleted of catecholamines by incubation 24 h at 37° C. followed by 72 h at ambient temperature. Eight levels of calibrators were prepared with concentrations doubling from 0.023 to 1.48 nmol/L for E, 0.098 nmol/L to 6.276 nmol/L for NE and from 0.039 nmol/L to 2.518 for DA. Medium (E: 0.472 nmol/L; NE: 1.355 nmol/L; DA: 0.923 nmol/L) and high (E: 2.308 nmol/L; NE: 10.798 nmol/L; DA: 4.416 nmol/L) concentration quality controls were purchased from Chromsystems (ref: 0010 and 0020). Low quality controls were in-house produced by spiking standard solutions of E, NE and DA at 0.08, 0.5 and 0.2 nmol/L, respectively. All calibrators and quality control samples were stored at −80° C. until use. Internal standard (IS) solutions containing d6-E, d6-NE and d4-DA at 100 μg/m1 concentration in 0.1 M HCl were stored at 4° C. The final working IS solutions for E and DA was adjusted to 12.5 ng/ml and NE to 125 ng/ml by dilution in ULC/MS water.

Samples Preparation

Microcentrifuge Tubes

50 to 250 μL of heparinised plasma (sample, calibrator or QC's) were delivered into a 0.5 ml Eppendorf microcentrifuge tubes, followed by the addition of 112.5 μl of 0.89 M Tris buffer pH 8.6 containing 0.56 mM Na₂S₂O₅ and 20 μl of a 125 ng/ml d6-NE, 12.5 ng/ml for d6-E and d4-DA into each well and 5 mg of activated aluminum oxide. The microcentrifuge tubes were mixed by rotation during 15 minutes on a wheel and after a quick spin in a microcentrifuge the plasma matrix was removed by aspiration using a fine-tipped glass pasteur pipettes. The activated aluminum oxide was washed with 3×250 μl of ULC/MS water. Then, 30 μl of a fresh solution of formic acid 2% in water (eluting buffer) was added and catecholamines and their stable isotope IS were eluted after 10 min of thorough shaking into polypropylene vials.

Microplates

50 to 250 μl of heparinised plasma (sample, calibrator or QC's) were delivered into a 96-well plate (see scheme below), followed by the addition of 112.5 μl of 0.89 M Tris buffer pH 8.6 containing 0.56 mM Na₂S₂O₅ and 20μl of a 125 ng/ml d6-NE, 12.5 ng/ml for d6-E and d4-DA into each well and 5 mg of activated aluminum oxide. The plate content was subsequently mixed on a vortex equipped with a Teflon adaptor to support the plate during 15 minutes.

Then, a vacuum was applied using an extraction plate manifold from Waters, Baden, CH (part #186001831) to discard plasma matrix. Wells were washed with 250 μl of ULC/MS water, vortexed for 1 min and discarded by vacuum. This operation was repeated 2 times. Then, 30 μl of a fresh solution of formic acid 2% in water (eluting buffer) was added and catecholamines and their stable isotope IS were eluted after 10 min of thorough shaking into a sample collection plate (Waters part # WAT058943).

Instrumentation

Microplate

Applicants used a specially designed plexiglass block for solid phase extraction (SPE) of plasma catecholamines. The system consists in a 96 well microplate (see FIG. 2). Each well is conical accordingly to the quotes on FIG. 2 to allow the creation of a vortex similar to that can be obtained by agitation of a microcentrifuge tube in a vortex. The top of the well may be closed by a plate sealer (Promega AG, ref 5701) during mixing or a cap foil of serigraphic quality (ref. Spondex WKU310). The activated alumina is retained in the bottom of the well by a 6 mm diameter filter in porous PTFE (Macherey-Nagel, Porafil membranes, pore size 3 μm, ref 670100013) and liquids are remaining above the filter since caps are inserted in the outlet (FIG. 3). The lower part of the filter (FIG. 4) is extended by a narrow tubing inserted in the plexiglass block to allow liquids to drain through a small insert itself extruding from the plate by a sticked outlet in PEEK (FIG. 5) to allow the generation of small droplets that will fall down the collection plate after elution of the catecholamines.

UPLC-MS/MS Conditions

The eluate from the UPLC system was connected to a tandem mass spectrometer Waters Acquity UPLC/TQD (Waters, Baden, CH). Separation of E, NE, DA and the deuterated IS was performed on a 1.9 μm particle size, 21 mm×100 mm Hypersil gold phenyl analytical column (Thermo Fisher Scientific, Basel, CH). The column temperature was set at 25-C. The injection volume was 6.7 μL. The isocratic mobile phase consists of 2% methanol and 98% of 50 mM formic acid in ULC/MS water. The flow rate was 0.5 mL/min and E, NE and DA and their deuterated analogues eluting at 0.64, 0.58 and 0.72 min, with a total run time of 2.0 min The eluent was introduced into the TQD mass spectrometer by positive ion electrospray ionization tandem mass spectrometry in the multiple reaction monitoring mode. Ion source conditions were optimised for the resulting ions arising from a facile loss of water for E=m/z 184 ([M+H]⁺), NE=m/z 152 ([M+H−H2O]⁺) and DA=m/z 154 ([M+H]⁺). Multiple-reaction monitoring transitions for E, NE and DA and their deuterated analogues along with mass detector settings are shown in Table 1. Instrument settings were optimised for maximum ion yield and were tuned individually for each biogenic amine (capillary voltage=0.63 kV, source temperature=150° C., desolvation temperature=450° C., and desolvation gas=900 L/h, dwell time was setted at 42 ms for each SRM transition).

TABLE 1

| | | Cone | Collision | |
|---|---|---|---|---|
| Analyte | MRM m/z pairs | (V) | (V) | Dwell (s) |
| Epinephrine | 184.0 > 166.0 | 15 | 13 | 0.042 |
| d6-Epinephrine | 190.0 > 172.1 | 17 | 11 | 0.042 |
| Norepinephrine | 152.0 > 107.0 | 30 | 18 | 0.042 |
| d6-Norepinephrine | 158.0 > 111.1 | 27 | 18 | 0.042 |
| Dopamine | 154.0 > 91.0 | 18 | 22 | 0.042 |
| d4-Dopamine | 158.0 > 141.0 | 16 | 12 | 0.042 |

Data Analysis

Chromatographic data were collected by monitoring UPLC elution times and ion-pairs corresponding to the precursor and product ion mass/charge ratios (m/z) of E, NE and DA and their respective IS. Data acquisition and control of the MS/MS system were performed using MassLynx™ v4.1 software with automated data processing by the QuanLynx Application Manager. The calibration curves were constructed based on the plotting of E, NE and DA to IS peak area ratios found (analyte/IS) for a given concentration. The calibration curves were calculated by least squares linear regression using a weighting factor of 1/concentration. Concentrations of E, NE and DA in unknown and QC's samples were determined using the response ratio from samples and the linear regression curve.

Method Validation

The developed method was validated for recovery, lower limit of quantification (LLOQ), linearity, range, accuracy, precision, selectivity, matrix effect, and carry-over in the spiked samples according to the US Food Drug Administration (FDA), Guidance for Industry:

Bioanalytical Method Validation. The method validation was performed with 0.25 ml plasma sample volume. The downsizing of samples volume to 0.05 and 0.1 ml has not followed the whole validation protocol but has been finally assessed on 3 distinct calibration curves including QC's in duplicate.

Linearity

The linearity of analytic method was performed using the 9 calibrators for each analyte in duplicate: 0.012 to 1.48 nmol/L for E, 0.049 nmol/L to 6.276 nmol/L for NE and from 0.020 nmol/L to 2.518 for DA. A constant amount of deuterated IS were included in all samples, 2.5 ng for NE-d6 and 0.25 ng for E-d6 and DA-d4.

Matrix Effect, Extraction Recovery and Process Efficiency

Matrix effect was qualitatively evaluated using the most current implemented technique proposed by Bonfiglio et al. [R. Bonfiglio, R.C. King, T. V. Olah, K. Merkle Rapid Commun. Mass Spectrom., 13 (1999), p. 1175]. Post-column infusion of catecholamines at 1 μg/m1 at a flow rate of 50 μl/min in parallel to injection of 6 different plasma sample extracts and chromatograms were plotted with the expected IS retention time. A decrease or increase in the MS signal at the retention time of the analytes and IS indicates the presence of a matrix effect. Matrix effect was then quantitatively assessed using the method proposed by Matuszewski et al. (B. K. Matuszewski, M. L. Constanzer, C. M. Chavezeng Anal. Chem., 75 (2003), p. 3019). Experiments were performed using catecholamines depleted heparin-lithium plasma spiked with low (close to the lower limit of quantification) (E: 0.08 nmol/1; NE: 0.50 nmol/1; DA: 0.2 nmol/L), medium (target values: E: 0.5 nmol/1; NE: 1.4 nmol/1; DA: 0.9 nmol/L) and high (target values: E: 2.3 nmol/1; NE: 10.8 nmol/1; DA: 4.4 nmol/L) catecholamine concentrations. Three different sets of solutions were prepared: 3 samples prepared in eluting buffer spiked with IS and the analytes (A), 6 different blank plasmas spiked with IS and the analytes added after extraction (B) and 6 different blank plasmas spiked with IS, and the analytes before extraction (C). Samples have been injected in triplicate and the mean peak area of the 3 determinations was used for the calculations. Matrix effect (% ME) was calculated with the ratio of peak areas from the post-extraction spiked analytes and the post-extraction spiked analytes in eluting buffer (ME=B/A)×100%. This equation means that no matrix effect is observed when % ME is equal to 100%. Values greater than 100% indicate a signal enhancement, whereas values lower than 100% indicate a trend in ionization suppression.

Extraction recovery (ER) was evaluated by the ratio of peak areas from the pre-extraction spiked plasmas and the post-extraction spiked plasmas (ER=C/B). Process efficiency (PE), which takes into account ME and ER, was calculated as the ratio of peak areas from the pre-extraction spiked plasmas and the post-extraction spiked eluting buffer (PE=C/A)×100%. The IS-normalized ME, ER and PE were calculated by dividing the result of the analytes by the result of the respective IS.

The inter-plasma variability of the parameters evaluated was assessed and expressed as relative standard deviation (RSD).

Lower Limit of Quantification (LLOQ) and Lower Limit of Detection (LLOD)

Calibrators from Chromsystems were half-diluted from 1.48 to 0.012 nM for E, 6.276 to 0.049 nM for NE and 2.518 to 0.02 nM for DA and the results from 5 experiments were analyzed to determine the LLOD and the LLOQ. The LLOD was determined as the concentration of compound with a signal to noise ratio of at least 5. The LLOQ was defined as the lower catecholamine concentration that allows a precision of 20%.

Precision and Accuracy

Precision was evaluated using three different concentrations of QC plasma (Low in-house prepared, medium and high concentration quality controls from Chromsystems as reported above). The 3 QC's described above are extracted 5 times and analyzed within the same chromatographic run (intra assay repetability) and extracted within distinct days (inter assay reproducibility). The precision determined at each concentration level should not exceed 15% of the coefficient of variation (CV) except for the LLOQ, where it should not exceed 20% of the CV.

Long term imprecision was also established in QC's run during 20 separate days. In addition, the accuracy of our measurement was investigated in two samples obtained from proficiency testing (Instand e.V., Düsseldorf, Germany) for catecholamines were also evaluated with the UPLC-MS/MS method.

Carry-Over

Carry-over was evaluated by preparing an aliquot of a QC sample and initially making three injections and subsequently three more injections of a blank incubation sample. Carry-over was expressed as the percentage difference between the mean analyte-to-IS area ratios for the blank incubation sample and QC sample. The percentage carry-over was calculated as follows: % Carry-over=(BL/QC)×100, where BL was the mean analyte-to-IS area ratio in the blank incubation sample and QC was the corresponding ratio in the QC sample. Carry-over was taken to be insignificant if less than 5%.

Method Comparison

For method comparison studies, plasma catecholamines (1 ml) were extracted using an in-house preparation of activated aluminum oxide and quantified by HPLC-ECD on a Coularray system (ESA-Dionex, Sunnyvale, Calif., USA) using a modified method of the RECIPE kit (ClinRep®, RECIPE Chemicals and Instruments GmbH, Munich, Germany) as previously reported. The LLOQ was 0.02 nmol/1 and inter-assay precisions (CV) were 14% and 7% for E, 7% and 5% for NE and 10 and 6.7% for DA for medium and high Chromsystems internal quality controls.

Plasma specimens from 64 patients were tested in duplicate for plasma catecholamines by the HPLC-EC and the UPLC-MS/MS method, and results were compared accordingly to the CLIA88 protocol. Results are reported as Deming regression curves and Altman Bland plots.

Reference Intervals

Heparinized plasmas collected from 120 healthy individuals in lying position were also measured to determine the reference intervals for catecholamines.

Effect of Sample Volume on the Linearity

Plasma calibration curves at 8 concentrations and controls were run with 50 and 100 pl and the ratio of analyte peak areas to IS peak areas were plotted and compared to results obtained with 250 pl normally used for validation.

Comparison of the Extraction Procedure between the Microplate Format and the Microcentrifuge Microtubes

250 μl of heparinised plasma calibrators with QC's were delivered into the microplate wells and the microcentrifuged tubes. The recoveries of activated aluminum oxide-extracted catecholamines and the concentrations of E, NE and DA in QC's samples were directly compared. The acquired data were statistically analyzed using the Analyse-it (version 2) add-on package for Microsoft Excel.

Results and Discussion

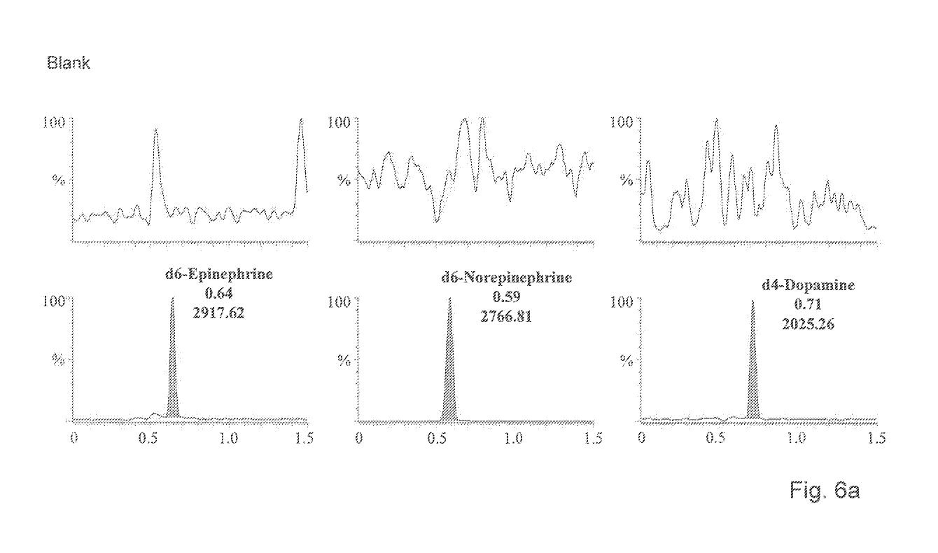

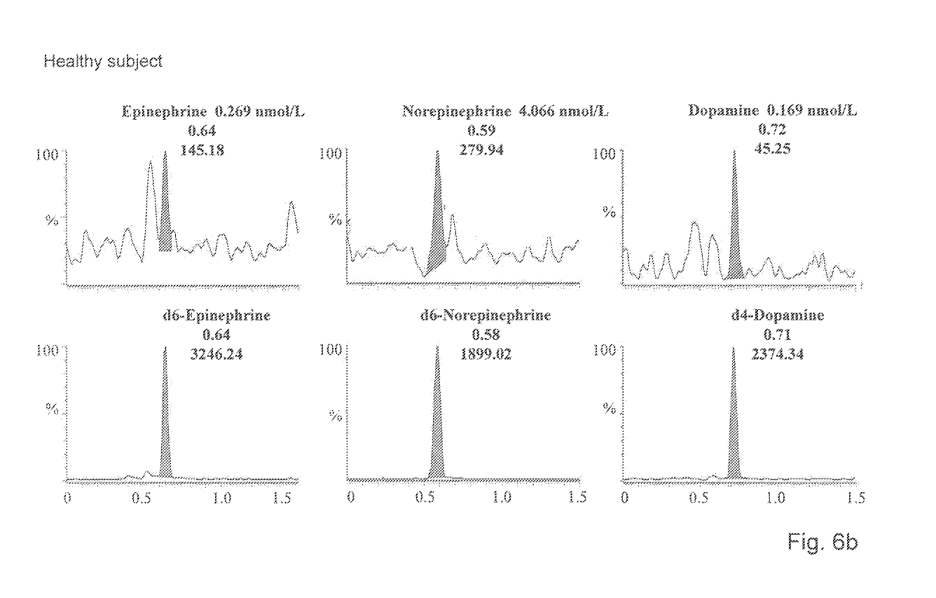

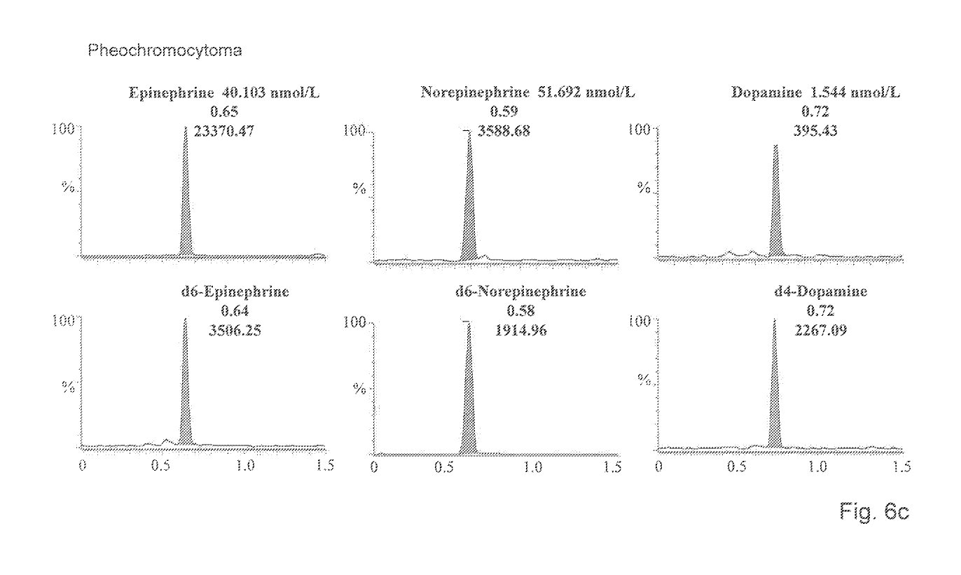

Chromatogram from a healthy subject along with an example of a patient with histologically confirmed pheochromocytoma are shown in FIG. 6 Complete chromatographic separation of all three catecholamines was performed in less than 1 min (E: 0.64 min, NE: 0.58 min and DA: 0.72 min) with the proposed gradient established with the mobile phase and the C18 column chosen for this application.

Linearity

Calibration curves and control samples were run with every batch of patient samples and a linear and reproducible response for E, NE and DA (r²=0.9914, 0.9813 and 0.9869) was observed over the concentration range defined for the 9 point calibration curve (Table 2). Plasma samples from a patient with histologically confirmed pheochromocytoma and elevated catecholamine concentrations was serially diluted with stripped serum and demonstrated linearity of the method (between 90 to 110% of the expected value) for E from 72.5 nmol/L to 0.142 nmol/L, NE from 102 nmol/1 to 0.1 nmol/L and DA from 11.6 nmol/L to 0.18 nmol/L (Table 3).

TABLE 2

| Analyte | Slope | Concentration range (nmol/L) | r2 |
|---|---|---|---|
| Epinephrine | 0.163 | 0.023-1.48 | 0.9914 |
| Norepinephrine | 0.036 | 0.098-6.276 | 0.9813 |
| Dopamine | 0.142 | 0.039-2.518 | 0.9869 |

TABLE 3

| | | Expected | Measured | |
|---|---|---|---|---|
| | | value | value | Percentage |
| Analyte | Dilution | (nmol/L) | (nmol/L) | of expected |
| Epinephrine | 1:1 | 72.480 | 72.480 | 100 |
| | 1:2 | 36.240 | 37.176 | 103 |
| | 1:4 | 18.120 | 18.408 | 102 |
| | 1:8 | 9.060 | 9.208 | 102 |
| | 1:16 | 4.530 | 4.438 | 98 |
| | 1:32 | 2.265 | 2.290 | 101 |
| | 1:64 | 1.133 | 1.160 | 102 |
| | 1:128 | 0.566 | 0.602 | 106 |
| | 1:256 | 0.283 | 0.288 | 102 |
| | 1:512 | 0.142 | 0.159 | 112 |
| | 1:1024 | 0.071 | 0.096 | 136 |
| | 1:2048 | 0.035 | 0.051 | 144 |
| | 1:4096 | 0.018 | 0.035 | 198 |
| Norepinephrine | 1:1 | 102.478 | 102.478 | 100 |
| | 1:2 | 51.239 | 50.375 | 98 |
| | 1:4 | 25.620 | 25.666 | 100 |
| | 1:8 | 12.810 | 12.521 | 98 |
| | 1:16 | 6.405 | 5.872 | 92 |
| | 1:32 | 3.202 | 2.690 | 84 |
| | 1:64 | 1.601 | 1.569 | 98 |
| | 1:128 | 0.801 | 0.613 | 77 |
| | 1:256 | 0.400 | 0.397 | 99 |
| | 1:512 | 0.200 | 0.184 | 92 |
| | 1:1024 | 0.100 | 0.095 | 95 |
| | 1:2048 | 0.050 | 0.000 | 0 |
| Dopamine | 1:1 | 11.569 | 11.569 | 100 |
| | 1:2 | 5.785 | 5.335 | 92 |
| | 1:4 | 2.892 | 2.715 | 94 |
| | 1:8 | 1.446 | 1.302 | 90 |
| | 1:16 | 0.723 | 0.661 | 91 |
| | 1:32 | 0.362 | 0.306 | 85 |
| | 1:64 | 0.181 | 0.182 | 101 |
| | 1:128 | 0.090 | 0.054 | 60 |
| | 1:256 | 0.045 | 0.000 | 0 |

Matrix Effect, Extraction Recovery and Process Efficiency Qualitative Matrix Effect

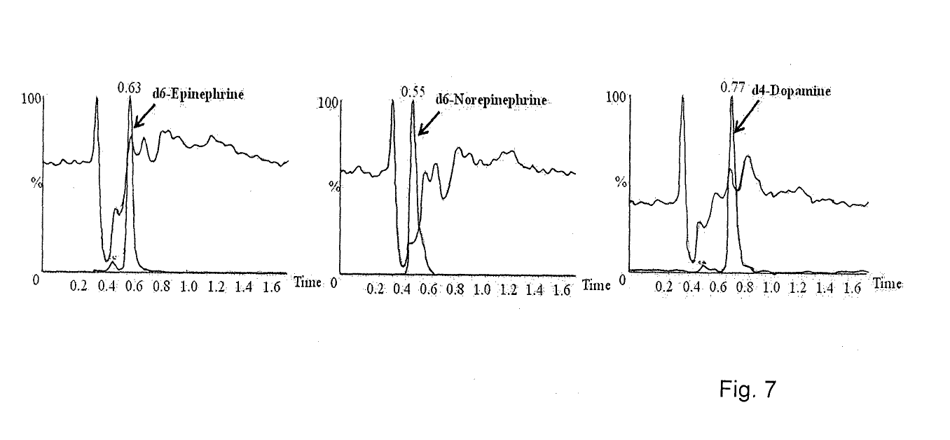

Activated aluminum oxide proves to be an efficient extraction method for catecholamines since no discernable ion suppression was observed at the retention times for E, NE and DA (FIG. 7).

Quantitative Recovery

The experiments were carried out at 3 catecholamine levels to take into account possible concentration-dependent recovery effects and were normalized to IS, calculated as the ratio of analyte area to that of its IS. Extraction recovery (RE) quantifying analyte losses associated to extraction by activated aluminum oxide was at (mean±SD) 48±0% for E, 48±11% for NE and 38±2% for DA was compensated by a positive matrix effect (ME) characterized by ionization enhancement at 178±6% for E, 212±28% for NE and 221±41% for DA. The resulting process efficiency (PE) measuring the net effect between extraction loss and positive ME was finally at 84±3% for E, 99±12% for NE and 84±12% for DA (Table 4).

TABLE 4

| | Concentration of | REa | MEb | PEc |
|---|---|---|---|---|
| | added analyte nmol/L | (%) | (%) | (%) |
| Epinephrine | 0.080 | 48 | 185 | 87 |
| | 0.500 | 48 | 177 | 85 |
| | 2.300 | 47 | 172 | 81 |
| | Mean (SD) | 48 (0) | 178 (6) | 84 (3) |
| Norepinephrine | 0.500 | 36 | 244 | 86 |
| | 1.400 | 50 | 203 | 101 |
| | 10.800 | 58 | 190 | 109 |
| | Mean (SD) | 48 (11) | 212 (28) | 99 (12) |
| Dopamine | 0.200 | 37 | 268 | 97 |
| | 0.900 | 39 | 197 | 76 |
| | 4.400 | 40 | 198 | 78 |
| | Mean (SD) | 38 (2) | 221 (41) | 84 (12) |

aRE: extration efficiency
bME: matrix effect
cPE: process efficency

Lower Limit of Quantification (LLOQ) and Lower Limit of Detection (LLOD)

The lowest limit of quantification (LLOQ), determined as the lowest concentration that produced a CV<20% was 0.05 nmol/L for E, 0.25 nmol/L for NE, and 0.15 nmol/L for DA. The lower limit of detection (LLOD), representing the absolute limit of detection that produced a signal-to-noise ratio of >5 was 0.03 nmol/L, 0.10 nmol/L, and 0.05 nmol/L for E, NE and DA respectively.

Precision and Accuracy

The intraassay and interassay CV% are summarized in Table 5. The day to day imprecision using 3 QC's at 3 levels ranged from 0.9 to 13.8% for E, 4.4 to 5.9% for NE and 8.5% to 12.3% for DA. Intra-assay precision ranged from 0.3 to 10.8% for E, 1.8 to 18.6% for NE and 4.1 to 27.8% for DA. To further validate the accuracy of the UPLC—MS/MS method, quality control samples distributed by Instand e. V. Quality Assurance Programme were measured and revealed excellent agreement with the median values reported by the Instand reporting group. Survey January 2012; 185#11: Target for HPLC-ECD in nmol/L (E: 3.259; NE: 12.939 and DA: 2.857) found with UPLC-MS/MS in nmol/L (E: 3.520; NE: 12.396 and DA: 3.516) with errors from the target for the 3 analytes between 4 and 23%. Survey January 2012; 185#12: Target for HPLC-ECD in nmol/L (E: 0.606; NE: 2.146 and DA: 0.594) found with UPLC-MS/MS in nmol/L (E: 0.681; NE: 2.246 and DA: 0.719) with errors from the target for the 3 analytes between 4 and 21%.

TABLE 5

| | | | Intra-Assay | | Inter-Assay | |
|---|---|---|---|---|---|---|
| | | Concen- | (n = 5) | | (n = 20) | |
| | | tration | Accu- | Preci- | Accu- | Preci- |
| | Sample | (nmol/ | racy | sion | racy | sion |
| | Type | L) | CV % | RSD % | CV % | RSD % |
| Epi- | QC-Low | 0.080 | 9.5 | 8.3 | 18.8 | 0.9 |
| nephrine | QC-Medium | 0.452 | 8.4 | −10.8 | 10.5 | −13.8 |
| | QC-High | 2.308 | 3.0 | 0.3 | 5.4 | −7.0 |
| Norepi- | QC-Low | 0.500 | 8.5 | −1.8 | 10.9 | −4.6 |
| nephrine | QC-Medium | 1.355 | 18.8 | −18.6 | 13.4 | −4.4 |
| | QC-High | 10.798 | 6.0 | 10.6 | 6.5 | −5.9 |
| Dopamine | QC-Low | 0.200 | 23.9 | −27.8 | 18.7 | −12.3 |
| | QC-Medium | 0.923 | 6.9 | −4.1 | 12.1 | −11.3 |
| | QC-High | 4.416 | 3.8 | −4.3 | 7.8 | −8.5 |

Carry-Over

Carry-over was measured using the high Level 2 from Chromsystems (target values: E: 2.308 nmol/1; NE: 10.798 nmol/1; DA: 4.416 nmol/L) after three injections followed by three more injections of a heparin-lithium plasma depleted of catecholamines sample. In the absence of any signal linked to E, NE and DA we concluded that these analytes passed the carryover test.

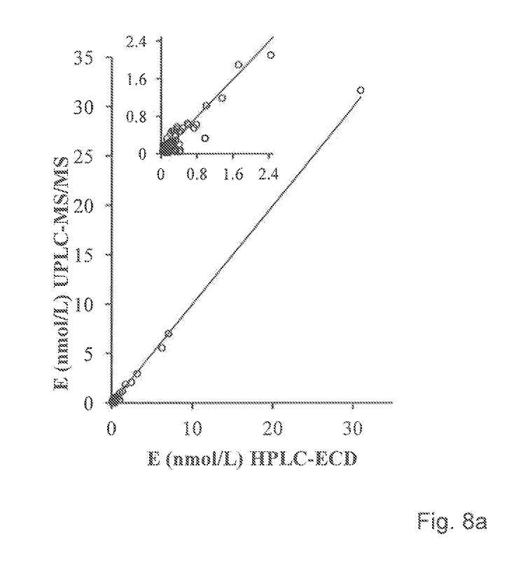

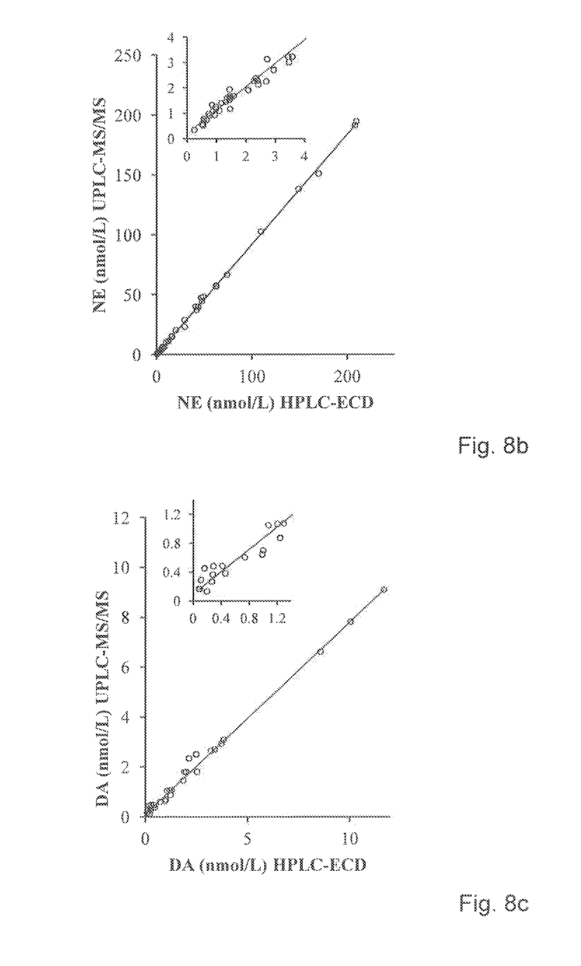

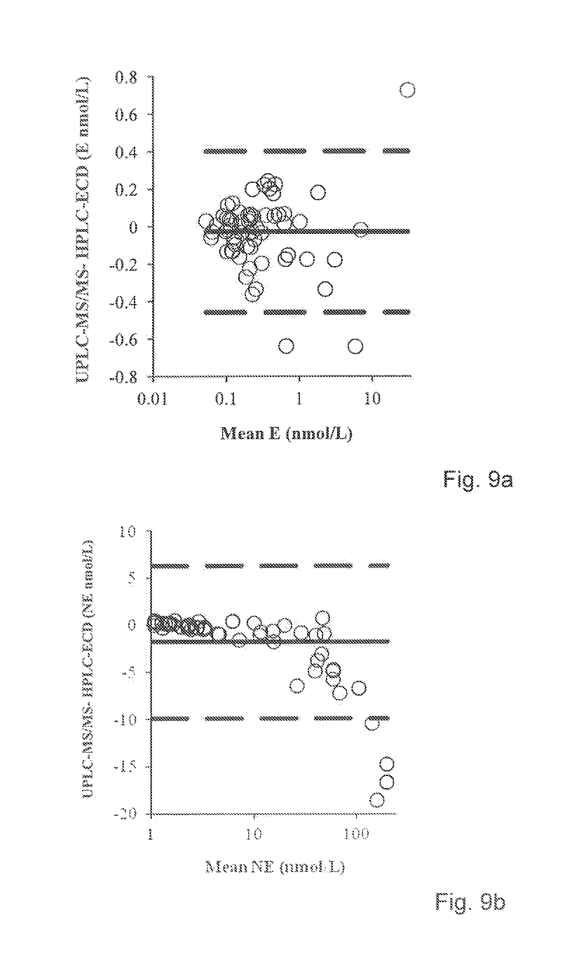

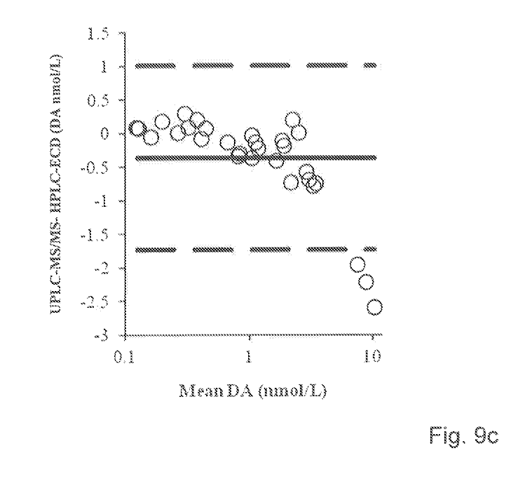

Method Comparison and Estimation of Reference Intervals Plasma catecholamines concentrations in plasma samples collected from patients screened for pheochromocytoma were determined by UPLC—MS/MS and compared with the results obtained by HPLC with electrochemical detection as routinely performed in our laboratory. Deming regression revealed the following curves equation: E UPLC-MS/MS =−0.05+1.02 E HPLC-EC (n=64); NE UPLC-MS/MS=0.22+0.92 NE HPLC-EC (n=64); DA UPLC-MS/MS=0.15+0.77 DA HPLC-EC (n=33) (FIG. 8A, B and C). Bland and Altman plots for the mean difference between HPLC-ECD and UPLC-MS/MS methods results in negative difference with mean difference of 0.03, 1.79 and 0.36 nmol/1 for E, NE and DA. NE concentrations higher than 20 nmol/L and DA concentrations higher than 3 nmol/L showed lower concentrations by UPLC-MS/MS than HPLC-ECD (FIG. 9A, B and C).

Reference intervals for plasma catecholamines for the UPLC—MS/MS method were based on the analysis of heparinized blood samples, collected in a recumbent position from a control group of 120 healthy volunteers (22-56 years). The distribution of values was skewed for all 3 catecholamines and therefore reference intervals were calculated after logarithmic transformation of the data. Reference intervals of 0.03 to 0.98 nmol/L for E, 0.63 to 4.51 nmol/L for NE and <LLOQ to 0.33 nmol/1 for DA were established in accordance with previously reported intervals for blood samples collected in a lying position.

Effect of Sample Volume on the Linearity

The slopes observed with were for 50 μl (E: 0.033; NE: 0.008 and DA: 0.031) and for 100 μl (E: 0.064; NE: 0.016 and DA: 0.061). Decreasing the sample volume results in lower sensitivity of the test for the three analytes (Table 6). Residual standard deviation lower than 20% for 50, 100 and 250 μl were found for E at 0.37, 0.18 and 0.045 nmol/L, for NE at 0.78, 0.39 and 0.19 nmol/L, and for DA at 0.315, 0.3 and 0.039 nmol/L.

TABLE 6

| | theoretical | observed | RSD | theoretical | observed | RSD | theoretical | observed | RSD |
|---|---|---|---|---|---|---|---|---|---|
| | value | value | % | value | value | % | value | value | % |
| Analyte | plasma volume 50 μl | | | plasma volume 100 μl | | | plasma volume 250 μl | | |
| Epinephrine | 1.480 | 1.379 | −6.8 | 1.480 | 1.507 | 1.8 | 1.480 | 1.473 | −0.5 |
| | 0.740 | 0.886 | 19.7 | 0.740 | 0.736 | −0.6 | 0.740 | 0.730 | −1.4 |
| | 0.370 | 0.326 | −12.0 | 0.370 | 0.364 | −1.6 | 0.370 | 0.385 | 4.1 |
| | | | | 0.185 | 0.170 | −8.4 | 0.185 | 0.165 | −10.8 |
| | | | | | | | 0.093 | 0.101 | 8.6 |
| | | | | | | | 0.046 | 0.060 | 29.7 |
| Norepinephrine | 6.276 | 5.908 | −5.9 | 6.276 | 6.192 | −1.3 | 6.276 | 6.477 | 3.2 |
| | 3.138 | 3.442 | 9.7 | 3.138 | 3.369 | 7.4 | 3.138 | 3.020 | −3.8 |
| | 1.569 | 1.595 | 1.7 | 1.569 | 1.477 | −5.9 | 1.569 | 1.482 | −5.6 |
| | 0.784 | 0.823 | 5.0 | 0.784 | 0.821 | 4.7 | 0.785 | 0.755 | −3.8 |
| | | | | 0.392 | 0.302 | −23.1 | 0.392 | 0.380 | −3.3 |
| | | | | | | | 0.196 | 0.243 | 23.9 |
| | | | | | | | 0.098 | 0.100 | 1.5 |
| Dopamine | 2.518 | 2.680 | 6.4 | 2.518 | 2.589 | 2.8 | 2.518 | 2.756 | 9.5 |
| | 1.259 | 1.101 | −12.5 | 1.259 | 1.251 | −0.6 | 1.259 | 1.132 | −10.1 |
| | 0.629 | 0.587 | −6.8 | 0.629 | 0.659 | 4.8 | 0.630 | 0.601 | −4.6 |
| | 0.315 | 0.352 | 11.7 | 0.315 | 0.243 | −23.0 | 0.315 | 0.259 | −17.9 |
| | 0.157 | 0.080 | −49.0 | 0.157 | 0.055 | −65.3 | 0.157 | 0.132 | −16.4 |
| | | | | 0.079 | 0.044 | −44.3 | 0.079 | 0.072 | −9.1 |
| | | | | | | | 0.039 | 0.047 | 18.2 |

Comparison of the Extraction Procedure between the Microplate Format and the Microcentrifuge Microtubes

A 8 point catecholamine calibration curve and low, medium and high QC's from Chromsystems were quantified the same day with microcentrifuge tubes and microplates by UPLC-MS/MS method. An excellent agreement between the two methods of extraction was found for medium and high QC's. Difference in concentrations found between microcentrifuge tubes and microplate were as follows (in %): Medium QC: E: 0, NE:-8 and DA: +13. High QC: E: −3, NE: +4 and DA: −2. Catecholamine concentrations in Low QC have been adjusted to be close to the LLOQ and differences were higher at E: −2, NE: −14 and DA: +35.

Extension to Catecholamine assay in Urines

Material and Methods

Microplates

250 μl of 1/200 diluted urine in 0.89 M Tris buffer pH 8.6 containing 0.56 mM Na₂S₂O₅ (calibrator from BioRad (Ref 195-5846) or QC's 1/200 diluted from Recipe (Level 1: 8820 and level 2: 8821)) were delivered into a 96-well plate, followed by the addition of 112.5μl of 0.89 M Tris buffer pH 8.6 containing 0.56 mM Na₂S₂O₅and 20μl of a 125 ng/ml d6-NE, 12.5 ng/ml for d6-E and d4-DA into each well and 5 mg of activated aluminum oxide. The plate content was subsequently mixed on a vortex equipped with a Teflon adaptor to support the plate during 15 minutes. Then, a vacuum was applied using an extraction plate manifold from Waters, Baden, CH (part # 186001831) to discard plasma matrix. Wells were washed with 250 μl of ULC/MS water, vortexed for 1 min and discarded by vacuum. This operation was repeated 2 times. Then, 30 μl of a fresh solution of formic acid 2% in water (eluting buffer) was added and catecholamines and their stable isotope IS were eluted after 10 min of thorough shaking into a sample collection plate (Waters part # WAT058943). The procedure used to quantify urine catecholamine followed the same approach than those used with plasma with the exception that one calibration point was used for urines. Biorad calibrator level: E: 875 nmol/L; NE: 1400 nmol/L; DA: 1775 nmol/L. Recipe Level 1 Target (−3S and +3S): E: 102 (81.9-122) nmol/L; NE: 336 (269-404) nmol/L; DA: 973 (777-1169) nmol/L. Recipe Level 2 Target (−3S and +3S): E: 204 (163-245) nmol/L; NE: 969 (774-1164) nmol/L; DA: 1470 (1176-1764) nmol/L

Results

The chromatograms were very clean and a good agreement was found between expected value and the concentration found in the Qc's as shown below: Recipe Level 1: E: 108 nmol/L; NE: 304 nmol/L; DA: 1176 nmol/LRecipe Level 2: E: 206 nmol/L; NE: 872 nmol/L; DA: 1741 nmol/L

Applicants concluded that the mass spectrometry method and the multi-well filter plate technology developed herein allow also determining catecholamines in urines.

### Example 2

Immunoextraction of Parathormone 1-34 (PTH1-34) from a Plasma Matrix Method

0.25 ml human plasma spiked with synthetic PTH1-34 (Bachem, CH) (0, 6.25, 12.5, 25 and 50 pM) and a triplicate of quality controls containing 20 pM of PTH1-34 were mixed with 80 pM of ¹³C PTH1-34 (Bachem, CH) that serves as a stable isotope internal standard and 5 microliters of beads coupled with anti-PTH monoclonal antibodies (Invitrogen, Norway). The mixture was filled in the wells of the multi-well filter plate of the invention and vortexed using the support for 60 min at 37 C. Then, the supernatant was drained by vacuum using the pump extractor and washed with 0.3 ml of water. The beads were then exposed to 50 microliter of acetic acid 2 M for 5 min at 37 C under thorough shaking. Finally, the eluate was recovered in collection Microplate (waters, WAT058943) coated with 0.5% BSA. The eluate was then desalted using Uptitips coated with C8 (Interchim, France) accordingly to kit recommendations. The PTH1-34 and its internal standard were finally eluted by 10 microliter of 50% acetonitrile diluted to 20% acetonitrile with water and 10 microliter were injected into an UPLC-MS/MS instrument (Xevo TQS, Waters). Quality controls were determined accordingly to the calibration curve and recoveries were compensated for peptide loss by using the ¹³C PTH1-34 internal standard.

Results

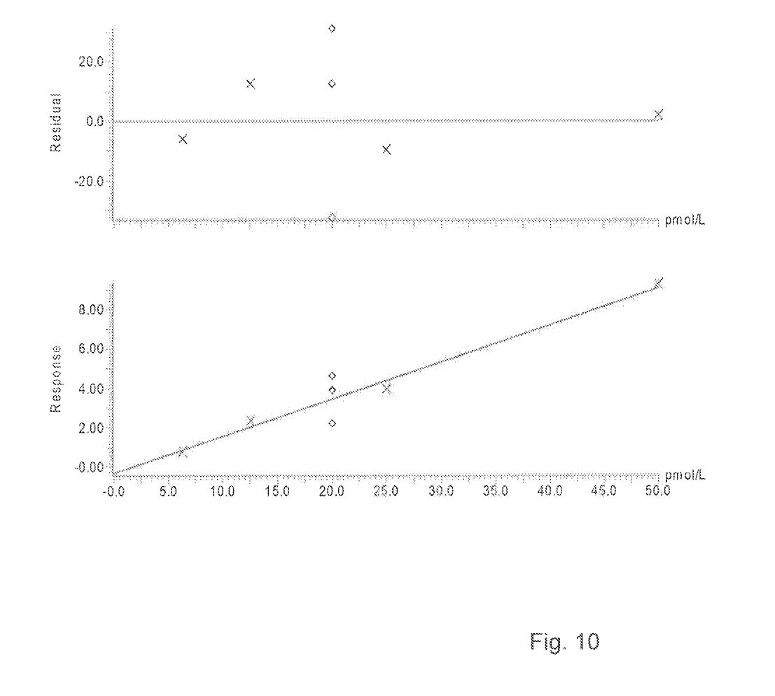

The +7 transition charged state used for PTH 1-34 (m/z=589.2→656.2) provides the best signal/noise ratio whereas the inventors used for ¹³C PTH1-34 the 5+state (m/z=825.6→159.1). FIG. 10 (lower panel) shows a nice calibration curve with a correlation coefficient r=0.994274 and a slope at 0.1875. The quality control (represented as circles) concentration found in the 3 samples is 13.5, 22.6 and 26.2 pmol/1 (mean=20.8 pmol/L and CV=30%). The residual values observed from the calibration curve varied from −9 to +12% indicating that they follow a 1/concentration curve type (upper panel).

In summary, the multi-well filter plate of the present invention allows to extract simultaneously and quantitatively picomolar concentrations of PTH1-34 from biological fluids and provide an alternative to tedious individual immunoextraction sample treatment.

1. A multi-well filter plate comprising a base plate having a plurality of wells therein, whereas each well of said plurality of wells is conical, with its wide end forming an inlet to said well on an upper side of said base plate and its narrow end being oriented towards a bottom side of said base plate, said well further comprising a filtered outlet for filtering and draining content out of said well.

2. The multi-well filter plate of claim 1, wherein said filtered outlet comprises a filter.

3. The multi-well filter plate of claim 1, wherein said filtered outlet comprises a membrane filter and a filter holder for holding said membrane filter outlet at the narrow end of said well.

4. The multi-well filter plate of claim 3, wherein the membrane filter is a microporous membrane filter selected from the group comprising nitrocellulose membrane, cellulose membrane, cellulose acetate membrane, polycarbonate membrane, polyvinylidene fluoride membrane and polysulfone membrane.

5. The multi-well filter plate of claim 1, wherein said filtered outlet comprises an outlet tube for forming droplets of a liquid flowing out of the well through the filtered outlet.

6. The multi-well filter plate of claim 1, wherein said filtered outlet further comprises a cap for preventing liquid from flowing out of the well through said filtered outlet.

7. The multi-well filter plate of claim 1, further comprising a cover for covering the inlets of said plurality of wells for preventing analytes from flowing out of said wells when said well is moved for mixing said analytes inside said plurality of wells.

8. A multi-well test apparatus comprising the multi-well filter plate of claim 1 and a feeding tray supporting said filter plate, said feeding tray having an inclined support surface comprising:
a drainage area from which liquid can be removed and an introduction area into which liquid can be supplied, said inclined support surface being inclined in a configuration to effect drainage of liquid from said introduction area to said drainage area, and
walls surrounding said inclined surface to enclose said inclined surface.

9. A kit comprising the multi-well filter plate according to claim 1 and chemical reagents.

10. The kit of claim 9, further comprising a cover.

11. The kit of claim 9, further comprising a collecting microplate that is located along the bottom of the multi-well filter plate, said waste tray element being adapted to receive filtrate from the multi-well filter plate.
